(19) United States
(12) Patent Application Publication
(10) Pub. No.: US 2026/0055361 A1
(43) Pub. Date: Feb. 26, 2026

(54) YEAST CULTIVATION METHOD AND MEDIUM THEREFOR

(71) Applicant: PURETURE, INC., New York, NY (US)

(72) Inventors: Yong kuk Park, Hwaseong-si (KR); Geunseo Yang, Centreville, VA (US)

(73) Assignee: PURETURE, INC., New York, NY (US)

(21) Appl. No.: 18/811,178

(22) Filed: Aug. 21, 2024

(51) Int. Cl. C12N 1/16

(52) U.S. Cl. CPC C12N 1/16

(57) ABSTRACT

A yeast cultivation medium and method of yeast cultivation are provided. The yeast cultivation medium can be an aqueous solution including molasses, rice water, and at least one hydrolytic enzyme, which can be a hydrolase. The yeast cultivation method can include adding a yeast strain to the yeast cultivation medium, heating the yeast-containing cultivation medium, and extracting the yeast from the cultivation medium.

### BACKGROUND OF THE INVENTION

The present invention relates to cultivation media and, more particularly, to a yeast cultivation medium adapted for improved yeast cultivation.

Yeast has a high content of digestible protein and has been used in the animal feed market for many years, either directly as feed material or as a feed additive. Yeast has potential use as a viable, scalable, plant-based source of functional and nutritional protein, having all necessary essential amino acids. Yeast protein may be used as a functional substitute for meat-and seafood-derived protein and may substitute for dairy-based protein (e.g., whey) on a one-to-one basis, reducing the need for dairy farming or livestock production and helping to eliminate world hunger Yeast protein also serves as an emulsifier. However, since yeast cells have an undesirable taste and smell, they are limited to certain applications, and except for some used for nutritional foods or fertilizers, most of them are discarded. For example, yeast generally has a bitter, astringent, acrid taste. Yeast propagation provides a stable source of animal-free protein for human nutrition that functions like animal protein. For example, yeast can provide a non-GMO plant-based casein to be used in alternatives to cheeses and other dairy products such as milk and yogurt.

Traditionally, commercial yeast production entails utilizing a yeast cultivation medium within a yeast cultivation process. The currently commercially available yeast cultivation medium contains molasses to provide carbon and nitrogen sources, water to provide a liquid cultivation medium, and antifoamer to suppress bubble generation. Silicon is a main component of antifoaming agents which can prevent the formation of bubbles, but disadvantageously can prevent the growth an development of yeast. Molasses is a source of energy and abundant inorganic substances, such as potassium, calcium, magnesium, sodium, iron, phosphorus, and manganese, as well as vitamins such as thiamine, betaine, and Vitamin B6 that help produce high-quality fermentation liquids for yeast propagation. The composition of molasses may vary depending upon the variations in sugar production methods, sources, and stages in the process from which the molasses may be extracted. Generally, molasses contains by weight about 20% sucrose, about 20% reducing sugars, about 10% ash, about 20% non-sugar organic materials, and about 20% water. The yeast cultivation process requires the components to be mixed and sterilized in an autoclave at 121° C. for 15 minutes prior to cultivation. A selected strain of yeast is added to the sterilized cultivation medium and held at 30° C. for 24 hours with 300 RPM stirring. To separate the cultivated yeast from the medium, the result is centrifuged for 10 minutes at 6000 RPM.

The commercial production of yeast is resource intensive, requiring large quantities of water and large amounts of energy for heat treatment during sterilization. As climate change and population growth strain resources such as water, techniques to re-utilize or recycle water are needed.

Rice water is a by-product of processing rice, which also requires large amounts of water. Rice water is rich in nutrients, such as carbohydrates, proteins, fats, vitamins and minerals, which could aid in the nutritional value of yeasts. Traditionally, rice water is not re-utilized or recycled, and is merely processed by wastewater treatment plants.

As can be seen, there is a need for an improved yeast cultivation medium that can integrate with existing yeast cultivation processes, reduce waste and environmental pollution, reduce undesirable tastes and smells, and improve resource usage.

### SUMMARY OF THE INVENTION

In one aspect of the present invention, a fermentation culture medium for producing yeast is provided. The fermentation culture medium comprises an aqueous solution that contains molasses, rice water, and at least one hydrolytic enzyme.

In another aspect of the present invention, a method for producing yeast is provided. The method includes adding a selected yeast culture to the novel fermentation culture medium provided.

These and other features, aspects and advantages of the present invention will become better understood with reference to the following drawings, description and claims.

### BRIEF DESCRIPTION OF THE DRAWINGS

FIG. 1 is a flowchart of an embodiment of a method of yeast cultivation utilizing a yeast cultivation medium, according to aspects of the present invention.

FIGS. 2A, 2B, 2C, and 2D are block diagrams of enzymatic treatment, according to aspects of the present invention;

FIG. 3A is an illustration of yeast cultivation utilizing prior art methods;

FIG. 3B is an illustration of yeast cultivation utilizing the method of FIG. 1 and the method of FIGS. 2A-2D;

FIG. 4A is an illustration of foam generated during yeast cultivation utilizing Potato Dextrose Broth as a medium;

FIG. 4B is an illustration of foam generated during yeast cultivation utilizing Molasses and Water as a medium; and

FIG. 4C is an illustration of no foam generated during yeast cultivation utilizing a rice water medium of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a yeast cultivation medium, which utilizes an upcycled material, rice water. In the present invention the rice water and an enzyme replace some or all fresh water and reduce or eliminate addition of an antifoamer to the composition.

The improved yeast cultivation medium when utilized in a yeast cultivation system reduces or eliminates bubble production during fermentation due to the starch content of rice water, thereby eliminating the need for an antifoaming agent. Additionally, the utilization of hydrolytic enzymes results in the absorption of bitter tasting peptides and malodorous components, which eliminates undesirable tastes and smells.

As used herein the term “rice water” and “rice wash water” are used interchangeably to describe starch-enriched water left over from washing, soaking, or cooking rice.

In embodiments, a yeast cultivation medium may include at least a carbon and nitrogen source and a starch-enriched water sourced from another manufacturing process.

The carbon and nitrogen source may be molasses, sugar, glucose, etc., for example.

The starch-enriched water source may be rice wash water, derived from processing rice. Rice water includes carbohydrates, proteins, fats, vitamins, and minerals and may be used to fortify or enrich yeast cultivated in a liquid cultivation medium. Advantageously, starch dissolved in the rice water can improve the nutritional value of yeast and can reduce or prevent foaming that occurs during prior art cultivation processes.

The yeast cultivation method may comprise hydrolytic enzymatic treatment. A hydrolytic enzyme breaks down starch to dextrin and maltooligosaccharides. The enzyme also converts amylose and amylopectin to starch. The process involves four enzymatic reactions, including cyclization, i.e., formation of a cyclic oligosaccharide; coupling, i.e., converting the cyclic oligosaccharide to a linear oligosaccharide; disproportionation, i.e., transferring the linearized oligosaccharide to an acceptor; and hydrolysis of the oligosaccharide chain. The inventors have surprisingly discovered that hydrolytic enzymatic treatment improves the taste and smell of yeast cultivated by the process described herein.

The inventors have surprisingly discovered that use of rice wash water with a content level of 1-10% of a hydrolytic enzyme reduces or eliminates foaming during yeast cultivation. Advantageously, the elimination of foaming without the need for antifoaming agents, containing silicon, prevents disruptions in yeast production and growth and allows for easier separation of yeast.

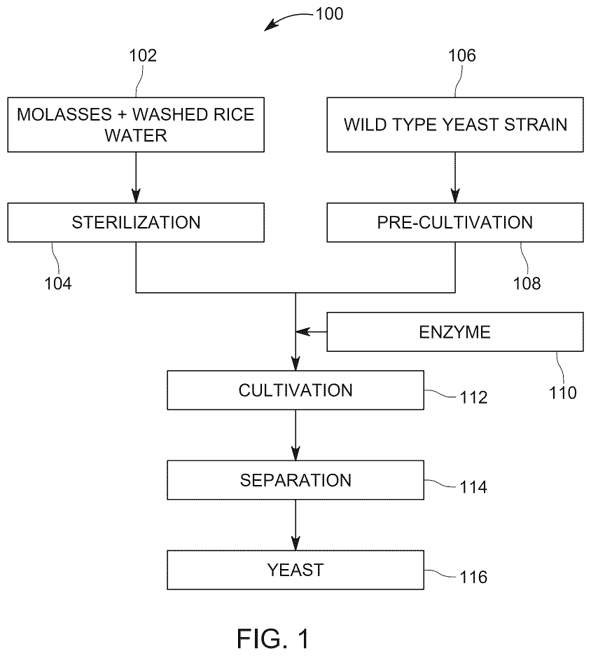

Referring now to the Figures, an embodiment of a yeast cultivation process 100 utilizing a yeast cultivation medium according to an embodiment of the present invention, is illustrated in FIG. 1. In step 102, the improved yeast cultivation medium is supplied to the yeast cultivation process. In embodiments, the improved yeast cultivation medium includes at least molasses and rice washed water.

In step 104, the improved yeast cultivation medium is sterilized to prevent contamination in the cultivation process. In embodiments, sterilization can be performed according to any suitable method known in the art. Sterilization can include introducing the improved yeast cultivation medium into a vessel and subjecting the medium to heat for a period of time. In embodiments, the vessel is an autoclave and the medium is subjected to a temperature of at least about 121° centigrade for a period of at least about 15 minutes. Advantageously, sterilization can remove impurities and lead to higher cultivation yields.

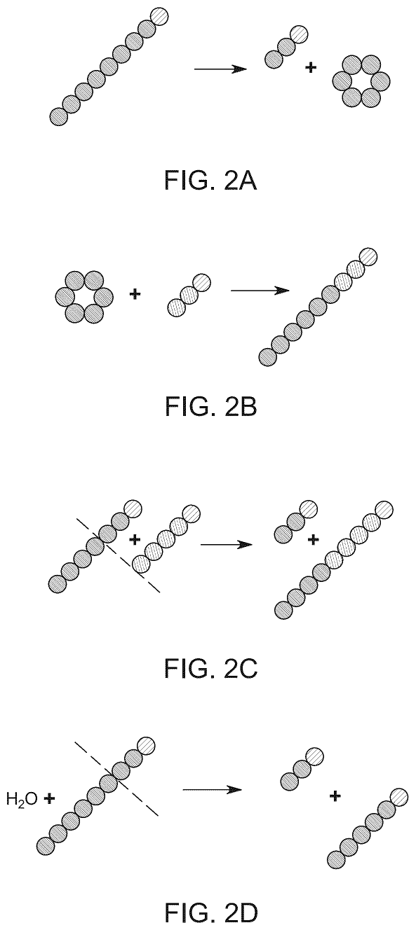

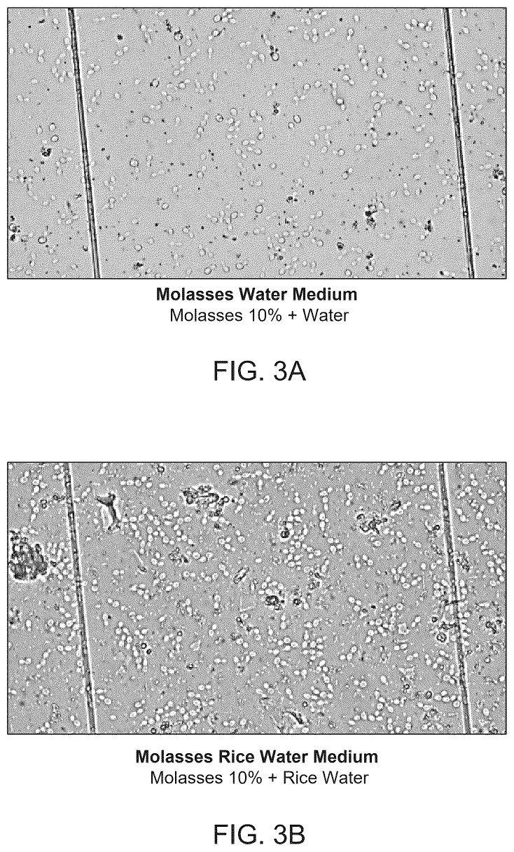

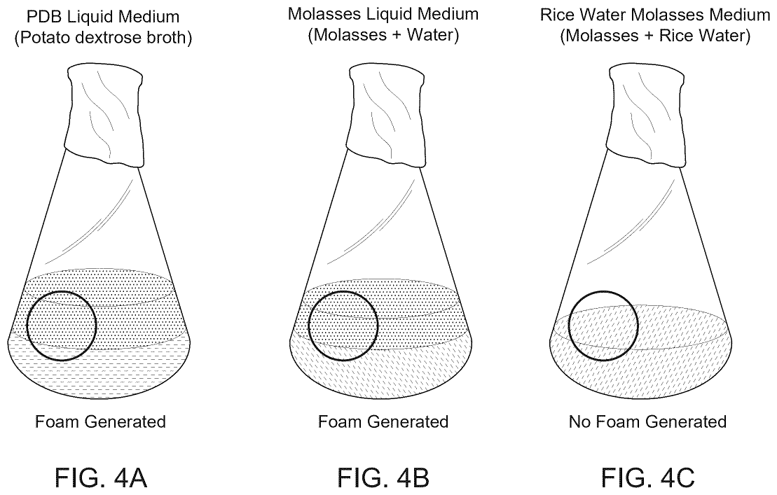

In step 106, a selected strain of yeast chosen. d. At step 108 pre-cultivation of the selected yeast strain can be performed according to any suitable method known in the art. At step 110, the pre-cultivated selected strain of yeast can be added to the sterilized cultivation medium along with at least one enzyme, and cultivation of yeast can proceed to form at least one cultivation composition. At step 112, as the cultivation proceeds, the enzyme catalyzes various reactions, converting amylose and amylopectin in the medium to starch and hydrolyzing the starch in the medium to produce dextrin and maltooligosaccharides, as shown in FIG. 2A (cyclization), 2B (coupling), 2C (disproportionation), and 2D (hydrolysis). In embodiments, the selected strain of yeast can be a wild-type yeast strain but is not so limited. Generally, the at least one enzyme is a hydrolytic enzyme, i.e., a hydrolase. The hydrolytic enzyme results in the production of inclusion compounds, such as Nitrogen compounds, from the starch, which absorb bitter-tasting peptides and malodorous components from the cultivation composition. Advantageously, the interactions of the inclusion compounds with the at least one cultivation composition improves the odor and taste of yeast cultivated by the method described herein, while simultaneously improving emulsification of the cultivated yeast. Furthermore, the inclusion of a hydrolytic enzyme and the resulting hydrolysis improves yeast growth during the cultivation process. The replacement of a molasses-fresh water medium (see FIG. 3A) with a molasses-rice water medium surprisingly results in a microscopically visible increase in yeast growth over 9 hours of cultivation. See FIG. 3B. Moreover, as shown in FIGS. 4A and 4B, prior art cultivation mediums (potato dextrose broth or molasses and water) both generate substantial foam during the cultivation of yeast. In contrast, the molasses-rice water medium in combination with an enzyme without an antifoaming agent surprisingly eliminates or minimizes foam generation, as illustrated in FIG. 4C.

In step 114, cultivated yeast can be separated from the at least one cultivation composition. Advantageously, the absence of antifoaming compounds aids in yeast separation of the present invention as there are no antifoaming compounds to bind to yeast causing difficulty with separation. In embodiments, separation can be achieved by introduction of the at least one cultivation into a secondary vessel and subjecting the compound to separation forces for a period of time. In embodiments, the secondary vessel can be a centrifuge which can produce separational forces through rotation. In step 116, yeast separated from the at least one cultivation compound can be recovered for utilization.

It should be understood, of course, that the foregoing relates to exemplary embodiments of the invention and that modifications may be made without departing from the spirit and scope of the invention as set forth in the following claims.

What is claimed is:

1. A fermentation culture medium for producing yeast, comprising:
an aqueous solution containing at least one carbon supply, rice water, and at least one hydrolytic enzyme.

2. The fermentation culture medium of claim 1, wherein the hydrolytic enzyme is a hydrolase.

3. The fermentation culture medium of claim 1, containing no antifoamer.

4. The fermentation culture medium of claim 1, wherein the at least one carbon supply is one of molasses, sugar, or glucose.

5. A method for cultivating yeast, comprising:
adding a selected yeast to the fermentation culture medium of claim 1;
heating the yeast-containing fermentation culture medium to a predetermined temperature;
maintaining the predetermined temperature for a selected time; and
separating the yeast from the fermentation culture medium.

6. The method of claim 4, further comprising sterilizing the fermentation culture medium before adding the yeast.
